SEQUENCE LISTING (12 sequences)

SEQ ID NO: 1 - RNA, 19 nt, Artificial Sequence
  Sense strand 1
gugugcacuucgcuucaca

SEQ ID NO: 2 - RNA, 21 nt, Artificial Sequence
  Sense strand 2
caccaugcaacuuuuucaccu

SEQ ID NO: 3 - RNA, 19 nt, Artificial Sequence
  Antisense strand 3
ugugaagcgaagugcacac

SEQ ID NO: 4 - RNA, 21 nt, Artificial Sequence
  Antisense strand 4
aggugaaaaaguugcauggug

SEQ ID NO: 5 - RNA, 19 nt, Artificial Sequence
  Sense strand 5
  misc_binding (1)..(2): The two nucleotide residues are connected by thiophosphate group
  modified_base (1)..(4): methoxy-modified
  misc_binding (2)..(3): The two nucleotide residues are connected by thiophosphate group
  modified_base (5)..(5): fluorine-modified
  modified_base (6)..(6): methoxy-modified
  modified_base (7)..(9): fluorine-modified
  modified_base (10)..(19): methoxy-modified
gugugcacuucgcuucaca

SEQ ID NO: 6 - RNA, 21 nt, Artificial Sequence
  Sense strand 6
  modified_base (1)..(6): methoxy-modified
  misc_binding (1)..(2): Adjacent nucleotide residues are connected by thiophosphate group
  misc_binding (2)..(3): Adjacent nucleotide residues are connected by thiophosphate group
  modified_base (7)..(7): fluorine-modified
  modified_base (8)..(8): methoxy-modified
  modified_base (9)..(11): fluorine-modified
  modified_base (12)..(21): methoxy-modified
crccaugcaacuuuuucaccu

SEQ ID NO: 7 - RNA, 21 nt, Artificial Sequence
  Antisense strand 7
  modified_base (1)..(1): methoxy-modified
  misc_binding (1)..(2): Adjacent nucleotide residues are connected by thiophosphate group
  modified_base (2)..(2): fluorine-modified
  misc_binding (2)..(3): Adjacent nucleotide residues are connected by thiophosphate group
  modified_base (3)..(5): methoxy-modified
  modified_base (6)..(6): fluorine-modified
  modified_base (7)..(7): methoxy-modified
  modified_base (8)..(9): fluorine-modified
  modified_base (10)..(13): methoxy-modified
  modified_base (14)..(14): fluorine-modified
  modified_base (15)..(15): methoxy-modified
  modified_base (16)..(16): fluorine-modified
  modified_base (17)..(21): methoxy-modified
  misc_binding (19)..(20): Adjacent nucleotide residues are connected by thiophosphate group
  misc_binding (20)..(21): Adjacent nucleotide residues are connected by thiophosphate group
ugugaagcgaagugcacacuu

SEQ ID NO: 8 - RNA, 23 nt, Artificial Sequence
  Antisense strand 8
  modified_base (1)..(1): methoxy-modified
  misc_binding (1)..(2): Adjacent nucleotide residues are connected by thiophosphate group
  misc_binding (2)..(3): Adjacent nucleotide residues are connected by thiophosphate group
  misc_feature (2)..(2): fluorine-modified
  modified_base (3)..(5): methoxy-modified
  misc_feature (6)..(6): fluorine-modified
  modified_base (7)..(7): methoxy-modified
  misc_feature (8)..(9): fluorine-modified
  modified_base (10)..(13): methoxy-modified
  misc_feature (14)..(14): fluorine-modified
  modified_base (15)..(15): methoxy-modified
  misc_feature (16)..(16): fluorine-modified
  misc_feature (17)..(23): methoxy-modified
  misc_binding (21)..(22): Adjacent nucleotide residues are connected by thiophosphate group
  misc_binding (22)..(23): Adjacent nucleotide residues are connected by thiophosphate group
aggugaaaaaguugcaugguguu

SEQ ID NO: 9 - RNA, 19 nt, Artificial Sequence
  Sense strand 9
  misc_binding (1)..(2): The two nucleotide residues are connected by thiophosphate group
  modified_base (1)..(4): methoxy-modified
  misc_binding (2)..(3): The two nucleotide residues are connected by thiophosphate group
  modified_base (5)..(5): fluorine-modified
  misc_binding (6)..(6): methoxy-modified
  modified_base (7)..(9): fluorine-modified
  modified_base (10)..(19): methoxy-modified
grgugcacuucgcuucaca

SEQ ID NO: 10 - RNA, 19 nt, Artificial Sequence
  Sense strand 10
  misc_binding (1)..(2): The two nucleotide residues are connected by thiophosphate group
  modified_base (1)..(4): methoxy-modified
  misc_binding (2)..(3): The two nucleotide residues are connected by thiophosphate group
  modified_base (5)..(5): fluorine-modified
  misc_binding (6)..(6): methoxy-modified
  modified_base (7)..(9): fluorine-modified
  modified_base (10)..(19): methoxy-modified
gurugcacuucgcuucaca

SEQ ID NO: 11 - RNA, 21 nt, Artificial Sequence
  Antisense strand 11
  misc_binding (1)..(2): The two nucleotide residues are connected by thiophosphate group
  modified_base (1)..(1): methoxy-modified
  modified_base (2)..(2): fluorine-modified
  misc_binding (2)..(3): The two nucleotide residues are connected by thiophosphate group
  modified_base (3)..(7): methoxy-modified
  modified_base (8)..(9): fluorine-modified
  misc_binding (10)..(13): methoxy-modified
  modified_base (14)..(14): fluorine-modified
  misc_binding (15)..(15): methoxy-modified
  modified_base (16)..(16): fluorine-modified
  modified_base (17)..(21): methoxy-modified
  misc_binding (19)..(20): The two nucleotide residues are connected by thiophosphate group
  misc_binding (20)..(21): The two nucleotide residues are connected by thiophosphate group
ugugargcgaagugcacacuu

SEQ ID NO: 12 - RNA, 21 nt, Artificial Sequence
  Sense strand 12
  modified_base (1)..(6): methoxy-modified
  misc_binding (1)..(2): Adjacent nucleotide residues are connected by thiophosphate group
  misc_binding (2)..(3): Adjacent nucleotide residues are connected by thiophosphate group
  modified_base (7)..(7): fluorine-modified
  modified_base (8)..(8): methoxy-modified
  modified_base (9)..(11): fluorine-modified
  modified_base (12)..(21): methoxy-modified
caccaugcaacuuuuucaccu

(19) United States
(12) Patent Application Publication
(10) Pub. No.: US 2023/0220386 A1
(43) Pub. Date: Jul. 13, 2023

(54) SIRNA CONJUGATE, DOUBLE-STRANDED SIRNA CONJUGATE, SALT THEREOF AND APPLICATION THEREOF

(71) Applicant: MEDSHINE DISCOVERY INC., Nanjing, Jiangsu (CN)

(72) Inventors: Ke AN, Shanghai (CN); Fei SUN, Shanghai (CN); Charles Z. DING, Shanghai (CN); Jian LI, Shanghai (CN); Shuhui CHEN, Shanghai (CN)

(21) Appl. No.: 17/781,876

(22) PCT Filed: Dec. 4, 2020
(86) PCT No.: PCT/CN2020/133982

§ 371 Date: Jun. 2, 2022

(30) Foreign Application Priority Data
Dec. 6, 2019 (CN) 201911243282.7
Jun. 10, 2020 (CN) 202010524584.8

(51) Int. Cl. C12N 15/113; A61K 31/713

(52) U.S. Cl. CPC C12N 15/113; A61K 31/713

(57) ABSTRACT

Provided is an application of a ribavirin derivative as an oligonucleotide embedded group. Specifically, disclosed is an application of r as a siRNA embedded group. Also provided is an r′-embedded siRNA conjugate, a double-stranded siRNA conjugate, a salt thereof, and an application thereof. The r′-embedded siRNA conjugate and the salt thereof can effectively reduce the S antigen content and E antigen content of the hepatitis B virus, providing an effective, feasible approach for the functional cure of chronic hepatitis B.

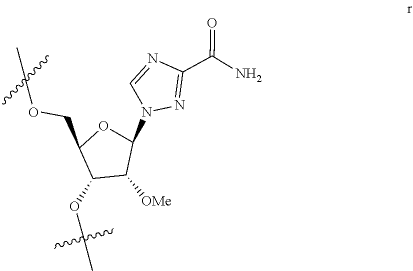 [SMILES: *OC[C@H]1O[C@@H](n2cnc(C(N)=O)n2)[C@H](OC)[C@@H]1O*]

The present application claims the right of the following priorities for: CN201911243282.7, filed on Dec. 6, 2019; CN202010524584.8, filed on Jun. 10, 2020.

### TECHNICAL FIELD

The present disclosure relates to use of a ribavirin derivative as an oligonucleotide embedded group, and specifically relates to use of r as a siRNA embedded group. The present disclosure also relates to an r-embedded siRNA conjugate, a double-stranded siRNA conjugate, a salt thereof, and use thereof.

Viral hepatitis B, referred to as hepatitis B, is a disease caused by the infection of the body by the hepatitis B virus (Hepatitis B Virus, referred to as HBV). Hepatitis B virus is a hepatotropic virus that mainly exists in liver cells and damages liver cells, causing inflammation, necrosis and fibrosis of liver cells. There are two types of viral hepatitis B, acute and chronic. In most adults with acute hepatitis B, self-healing happens through their intrinsic immunity mechanism. However, chronic hepatitis B (CHB) had became a great challenge for global health care and a major cause of chronic liver disease, cirrhosis and liver cancer (HCC) (Edward J. G., et al., The oral toll-like receptor-7 agonist GS-9620 in patients with chronic hepatitis B virus infection. Journal of Hepatology (2015); 63: 320-328). It is estimated that 2 billion people worldwide were infected with chronic hepatitis B virus, more than 350 million people had progressed to hepatitis B, and nearly 600,000 people died each year from complications of chronic hepatitis B (Edward J. G., et al., The oral toll-like receptor 7 agonist GS-9620 in patients with chronic hepatitis B virus infection. Journal of Hepatology (2015)). China was a high incidence area of hepatitis B, with a large number of accumulated patients and severe harmness. According to the data, there were about 93 million people infected with hepatitis B virus in China, and about 20 million patients were diagnosed with chronic hepatitis B, of which 10%-20% could progress into cirrhosis and 1% could progress into liver cancer. (Zhang Chunhong, The application of interferon in the treatment of hepatitis B. Chinese Medicine Guide (2013); 11: 475-476.)

The key to the functional cure of hepatitis B is to eliminate HBsAg (hepatitis B surface antigen) and produce surface antibodies. Quantitative HBsAg is a very important biomarker. In patients with chronic infection, the decrease of HBsAg and seroconversion are rarely observed, which are endpoints of current treatment.

At present, the anti-HBV drugs currently approved for marketing are mainly immunomodulators (interferon-α and peginterferon-α-2α) and antiviral therapeutic drugs (lamivudine, adefovir dipivoxil, entecavir, telbivudine, tenofovir, clevudine, etc.). Wherein, antiviral therapeutic drugs belong to nucleotide drug category, and the mechanism of action is to inhibit the synthesis of HBV DNA, and cannot directly reduce the level of HBsAg. As with prolonged treatment, nucleotide drugs display a similar HBsAg clearance rate to natural observation (Janssen et al. Lancet (2005), 365, 123-129; Marcellin et al. N. Engl. J. Med. (2004), 351, 1206-1217; Buster et al. Hepatology (2007), 46, 388-394.).

Clinical therapies have been available to reduce HBsAg, but the therapeutic effect is unsatisfactory. Therefore, if the gene expression of the virus can be silenced at the gene level, and the generation and replication of HBV can be blocked, especially the generation of HBsAg and HBeAg (S antigen and E antigen of hepatitis B) can fundamentally reduce virus metabolism and invasion to liver cells. Based on the mechanism of RNA interference (RNAi), small interfering RNA (siRNA) is able to inhibit or block the expression of the target gene in a sequence-specific way, and play an inhibitory effect from the level of mRNA translation to protein, thus achieving the purpose of treating diseases (WO2016077321, WO2018195165). This is the most ideal treatment method for hepatitis B, which requires the stabilization modification of siRNA and the auxiliary targeting of the corresponding delivery system to the target organs and cells, so as to improve the metabolic stability. However, the current siRNA can not effectively reduce the S antigen content and E antigen content of the hepatitis B virus.

### Content of the Present Invention

The present disclosure provides use of r as an oligonucleotide embedded group, and the r is

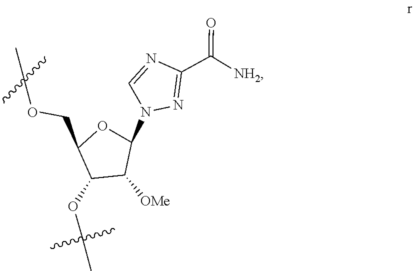

wherein, the oligonucleotide is a nucleotide sequence containing 10 to 50 nucleotides or nucleotide base pairs, and the oligonucleotide is able to inhibit or block gene expression.

In some embodiments of the present disclosure, in the use, wherein, the gene is HBV gene.

In some embodiments of the present disclosure, in the use, wherein, the oligonucleotide is siRNA.

In some embodiments of the present disclosure, in the use, wherein, the siRNA comprises sense strand and antisense strand.

In some embodiments of the present disclosure, in the use, wherein, the r is only embedded in the sense strand of the siRNA.

In some embodiments of the present disclosure, in the use, wherein, the r is only embedded in the antisense strand of the siRNA.

In some embodiments of the present disclosure, in the use, wherein, the r is embedded in the sense strand and antisense strand of the siRNA.

In some embodiments of the present disclosure, in the use, wherein, the sense strand of the siRNA comprises a sequence as shown in SEQ ID NO: 5 or SEQ ID NO: 12.

In some embodiments of the present disclosure, in the use, wherein, the antisense strand of the siRNA comprises a sequence as shown in SEQ ID NO: 7 or SEQ ID NO: 8.

In some embodiments of the present disclosure, in the use, wherein, the sense strand and the antisense strand of the siRNA respectively comprises a sequence as shown in SEQ ID NO: 5 and SEQ ID NO: 7, or the sense strand and the antisense strand of the siRNA respectively comprises a sequence as shown in SEQ ID NO: 12 and SEQ ID NO: 8.

In some embodiments of the present disclosure, in the use, wherein, the sense strand of the siRNA comprises an embedded group r.

In some embodiments of the present disclosure, in the use, wherein, the antisense strand of the siRNA comprises an embedded group r.

In some embodiments of the present disclosure, in the use, wherein, the sense strand and the antisense strand of the siRNA both comprise embedded group r.

In some embodiments of the present disclosure, in the use, wherein, the sense strand comprises a sequence as shown in SEQ ID NO: 6.

In some embodiments of the present disclosure, in the use, wherein, the antisense strand comprises a sequence as shown in SEQ ID NO: 8.

In order to solve the technical problems of low metabolic stability of siRNA, poor therapeutic effect and off-target effect in gene silencing in the prior art, the present disclosure provides a double-stranded siRNA conjugate, salt thereof and use thereof.

In order to solve the above technical problems, the first technical solution of the present disclosure is: 1) to provide a siRNA conjugate, which is characterized in that, the structure thereof is shown in formula (I):

S-L (I)

wherein, the nucleotide sequence of S is shown in SEQ ID NO: 6, SEQ ID NO: 9 or SEQ ID NO: 10, and the L is shown in formula (II):

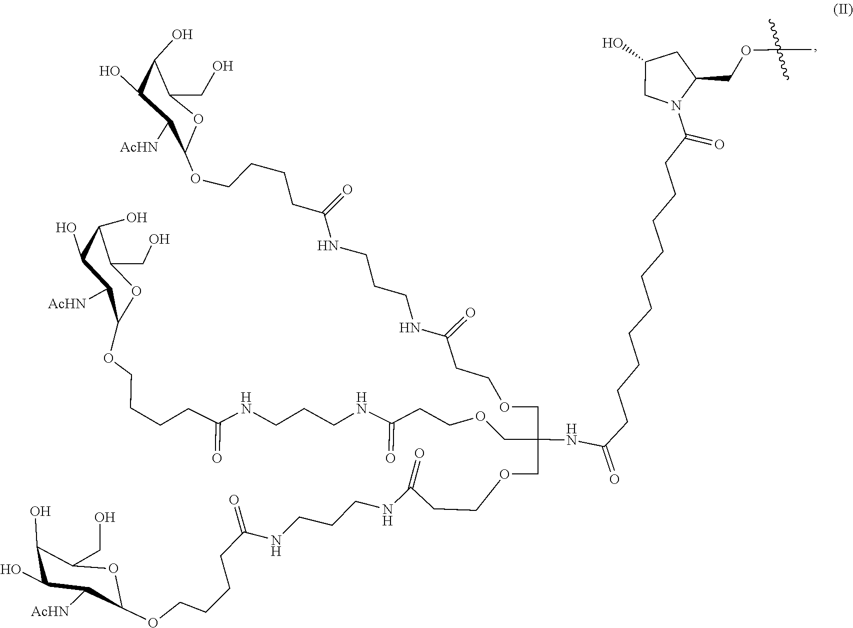

and the L is connected to the 3′ end of the nucleotide sequence of the S.

2) to provide a siRNA conjugate, and the structure thereof is shown in formula (I):

S-L (I)

wherein, the nucleotide sequence of S is shown in SEQ ID NO: 6, and the L is shown in formula (II):

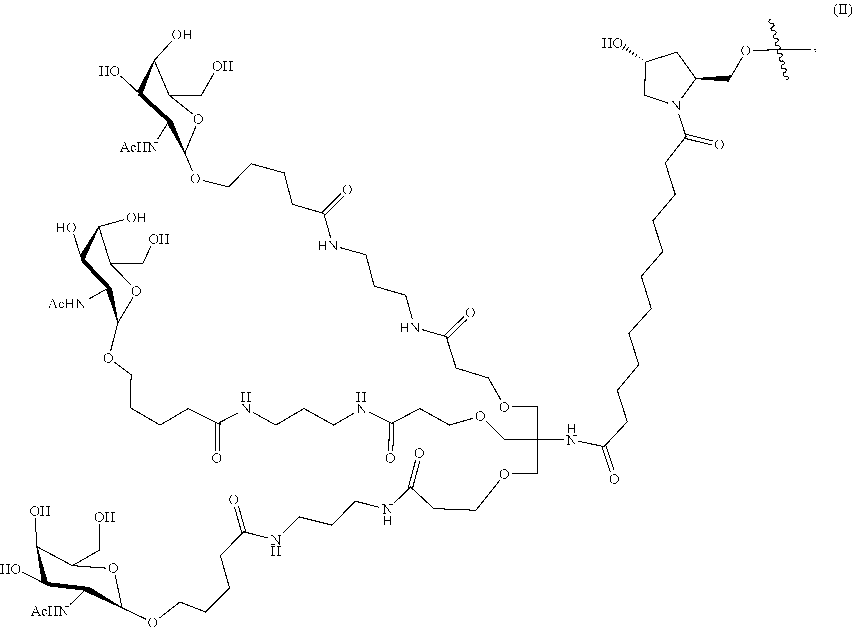

and the L is connected to the 3′ end of the nucleotide sequence of the S. Preferably, the thiophosphate moiety of the siRNA conjugate comprises (R)- and (S)-enantiomers, diastereomers, and/or racemic mixtures thereof.

In order to solve the above technical problems, the second technical solution of the present disclosure is to provide a salt of the siRNA conjugate.

In order to solve the above technical problems, the third technical solution of the present disclosure is: 1) to provide a double-stranded siRNA conjugate, which is characterized in that, the double-stranded siRNA conjugate comprises a sense strand and an antisense strand, and the sense strand is the above siRNA conjugates.

2) to provide a double-stranded siRNA conjugate, wherein, the double-stranded siRNA conjugate comprises a sense strand and an antisense strand, and the nucleotide sequence of the sense strand is shown in SEQ ID NO: 6, SEQ ID NO: 9 or SEQ ID NO: 10.

3) to provide a double-stranded siRNA conjugate, wherein, the double-stranded siRNA conjugate comprises a sense strand and an antisense strand, and the nucleotide sequence of the sense strand is shown in SEQ ID NO: 6.

Preferably, the nucleotide sequence of the antisense strand is shown in SEQ ID NO: 7, SEQ ID NO: 8 or SEQ ID NO: 11.

Preferably, the nucleotide sequence of the antisense strand is shown in SEQ ID NO: 8.

Preferably, the thiophosphate moiety of the double-stranded siRNA conjugate comprises (R)- and (S)-enantiomers, diastereomers, and/or racemic mixtures thereof.

In order to solve the above technical problems, the fourth technical solution of the present disclosure is to provide a salt of the double-stranded siRNA conjugate.

Preferably, the salt of the siRNA conjugate and the salt of the double-stranded siRNA conjugate comprise a base addition salt and an acid addition salt.

More preferably, the base addition salts comprise sodium, potassium, calcium, ammonium, organic amine or magnesium salts; the acid addition salts comprise inorganic acid salts and organic acid salts; preferably, the inorganic acid comprises, for example, hydrochloric acid, hydrobromic acid, nitric acid, carbonic acid, bicarbonate, phosphoric acid, monohydrogen phosphate, dihydrogen phosphate, sulfuric acid, hydrogen sulfate, hydroiodic acid, phosphorous acid, and the organic acid comprises, for example, acetic acid, propionic acid, isobutyric acid, maleic acid, malonic acid, benzoic acid, succinic acid, suberic acid, fumaric acid, lactic acid, mandelic acid, phthalic acid, benzenesulfonic acid, p-toluenesulfonic acid, citric acid, tartaric acid, and methanesulfonic acid.

In order to solve the above technical problems, the fifth technical solution of the present disclosure is to provide a method for preparing the above double-stranded nucleic acid-like biological oligomer; preferably, the method comprises the following steps:

A. synthesizing compound 1 from (2S,3R,4R,5R,6R)-3-acetamido (acetoxymethyl)tetrahydro-2H-pyran-2,4,5-triyl triacetate (formula 1-1);

B. connecting the compound 1 with natural nucleotide and modified nucleotide by chemical bonds to obtain the nucleic acid-like conjugate, preferably the chemical bonds are phosphate group or thiophosphate group.

In order to solve the above technical problems, the sixth technical solution of the present disclosure is to provide use of the siRNA conjugate, the salt of the siRNA conjugate, the double-stranded siRNA conjugate, or the double-stranded siRNA conjugate in the manufacture of a medicament for the treatment of viral hepatitis B.

In order to solve the above technical problems, the seventh technical solution of the present disclosure is to provide a method for treating viral hepatitis B, comprising administering the siRNA conjugate, the salt of the siRNA conjugate, the double-stranded siRNA conjugate or the salt of the double-stranded siRNA conjugate to patients or subjects in need thereof.

On the basis of conforming to the common sense in the field, the above-mentioned preferred conditions can be arbitrarily combined to obtain the preferred embodiments of the present disclosure.

Unless otherwise specified, the following terms and phrases when used herein have the following meanings. A specific term or phrase should not be considered indefinite or unclear in the absence of a particular definition, but should be understood in the ordinary sense. When a trade name appears herein, it is intended to refer to its corresponding commodity or active ingredient thereof.

The term “connect” used in the present disclosure, when indicating the connection between two molecules, means that two molecules are connected by covalent bonds or two molecules are associated by non-covalent bonds (e.g., hydrogen bonds or ionic bonds).

The “oligonucleotide” of the present disclosure is a nucleotide sequence containing 10 to 50 nucleotides or nucleotide base pairs. In some embodiments of the present disclosure, the oligonucleotide has a nucleobase sequence that is at least partially complementary to a coding sequence in a target nucleic acid or or target genes expressed in cell. The nucleotides can optionally be modified. In some embodiments of the present disclosure, after the oligonucleotide is delivered to the cell expressing the gene, the oligonucleotide is able to inhibit or block the gene expression in vitro or in vivo. “Oligonuclotide” includes, but is not limited to, single-stranded oligonucleotide, single-stranded antisense oligonucleotide, short interfering RNA (siRNA), double-stranded RNA (dsRNA), microRNA (miRNA), short hairpin RNA (shRNA), ribozyme, interfering RNA molecule and Dicer enzyme substrate.

The term “single-stranded oligonucleotide” of the present disclosure refers to a single-stranded oligonucleotide with a sequence at least partially complementary to the target mRNA, which is able to hybridize with the target mRNA under physiological conditions of mammals (or equivalent in vitro environment) by hydrogen bonding. In some embodiments of the present disclosure, the single-stranded oligonucleotide is a single-stranded antisense oligonucleotide.

The short interfering RNA (siRNA) of the present disclosure is a kind of RNA molecule with a length of 20-25 base pairs, similar to miRNA, and operates in the RNA interference (RNAi) pathway, which interferes with the translation of mRNA of a specific gene complementary to the nucleotide sequence, leading to mRNA degradation. The short interfering RNA (siRNA) of the present disclosure comprises double-stranded siRNA (comprising sense strand and antisense strand) and single-stranded siRNA (e.g., comprising only antisense strand).

The “inhibition” of the present disclosure, when referring to the expression of a given gene, means that the gene expression is reduced when the cell, cell group or tissue is treated with the oligonucleotide of the present disclosure, compared with the cell, cell group or tissue that has not been treated in this way.

The “sequence” or “nucleotide sequence” in the present disclosure refers to the order or sequence of nucleobases or nucleotides described by a series of letters using standard nucleotide nomenclature.

In the present disclosure, HBV gene refers to a gene whose DNA sequence is shown in Genbank Registration No. NC_003977.1.

The siRNA of the present disclosure contains a nucleotide group as a basic structural unit, and as known to those skilled in the art, the nucleotide group contains a phosphate group, a ribose group and a base, which will not be repeated here. Each nucleotide in the siRNA is independently an unmodified nucleotide. The double-stranded siRNA of the present disclosure contains a sense strand and an antisense strand.

Each nucleotide in the sense and antisense strands is each independently a modified or unmodified nucleotide. In the context of the present disclosure, unless otherwise specified, “conjugation” means that two or more chemical moieties, each having a specific function, are connected to each other in a covalent connection manner; accordingly, “conjugate” refers to a compound formed by covalent connection between the various chemical moieties. Further, “siRNA conjugate” refers to a compound formed by covalently connecting one or more chemical moieties with a specific function to an siRNA. Hereinafter, the siRNA conjugate of the present disclosure is sometimes simply referred to as “conjugate”. According to the context, siRNA conjugate should be understood as the general term of siRNA conjugates, the first or second siRNA conjugates, or siRNA sense chain conjugates or siRNA antisense strand conjugates.

In some embodiments, the conjugated group can be connected to the phosphate group, the hydroxyl group at the 2′-position, or the base of the nucleotide. In some embodiments, the conjugated group can be connected to the hydroxyl group at the 3′-position, and the nucleotides are connected by 2′-5′ phosphodiester bonds. When the conjugated group is connected to the end of the siRNA strand, the conjugated group is usually connected to the phosphate group of the nucleotide; when the conjugated group is connected to the internal sequence of the siRNA, the conjugated group is usually connected to a ribose ring or base. Various connection modes can be referred to: Muthiah Manoharan et. al. siRNA conjugates carrying sequentially assembled trivalent N-acetylgalactosamine linked through nucleosides elicit robust gene silencing in vivo in hepatocytes. ACS Chemical biology, 2015, 10(5): 1181-7.

In some embodiments, the siRNA and the conjugated group can be connected by acid-labile or reducible chemical bonds, which can be degraded in the acidic environment of the cellular endosome, thereby leaving the siRNA in a free state. For non-degradable conjugation modes, the conjugated group can be connected to the sense strand of siRNA, thereby minimizing the effect of conjugation on siRNA activity.

The nucleotide sequences of the sense strand and the antisense strand are at least partially reverse complementary to form a double-stranded region, and the nucleotide sequences of the sense strand and the antisense strand are of similar or equal length, and the length difference is no more than 3 nucleotides when the length is similar. These nucleotide differences do not significantly reduce the target gene inhibitory ability of the double-stranded siRNA conjugates, and siRNA conjugates comprising nucleotide differences are also within the scope of the present disclosure. In this context, “positional correspondence” means being at the same position in the nucleotide sequence from the same end of the nucleotide sequence. For example, the first nucleotide at the 3′ end of the nucleotide sequence of the sense strand is the nucleotide at the position corresponding to the first nucleotide at the 3′ end of SEQ ID NO: 1.

In some embodiments, the nucleotide sequence of the sense strand and the nucleotide sequence of the antisense strand are substantially reverse complementary, essentially reverse complementary, or fully reverse complementary; the substantially reverse complementary refers to the existence of no more than 3 base mismatches between two nucleotide sequences; the essentially reverse complementary refers to the existence of no more than 1 base mismatch between the two nucleotide sequences; fully reverse complementary means that there are no mismatches between the two nucleotide sequences. In addition, the length of the sense strand and the antisense strand are the same or different, and the length of the sense strand is 19-23 nucleotides, and the length of the antisense strand is 20-26 nucleotides. In this way, the length ratio of the sense strand and the antisense strand of the siRNA or siRNA conjugate provided by the present disclosure can be 19/20, 19/21, 19/22, 19/23, 19/24, 19/25, 19/20 26, 20/20, 20/21, 20/22, 20/23, 20/24, 20/25, 20/26, 21/20, 21/21, 21/22, 21/23, 21/24, 21/25, 21/26, 22/20, 22/21, 22/22, 22/23, 22/24, 22/25, 22/26, 23/20, 23/21, 23/22, 23/23, 23/24, 23/25 or 23/26. In some embodiments, the length ratio of the sense and antisense strands of the siRNA or siRNA conjugate is 19/21, 21/21, 21/23, or 23/25.

In the present disclosure, unless otherwise specified, uppercase letters C, G, U and A indicate the base composition of nucleotides. The lowercase letters c, g, u, and a indicate that the nucleotides represented by the corresponding uppercase letters are modified by methoxy; the underline indicates that the nucleotides represented by the uppercase letters are modified by fluoro; separation dot “•” indicates that there is a thiophosphate group linkage between two nucleotide residues adjacent to the left and right of the separation dot “•”. For example, “ag” indicates that the a and g residues are connected by a thiophosphate group.

The nucleotides modified by fluoro in the present disclosure indicate nucleotides formed by substituting the hydroxyl group at the 2′-position of the ribosyl with fluorine, and the nucleotides modified by methoxy indicate nucleotides formed by substituting the hydroxyl group at the 2′-position of the ribosyl with methoxy.

The “modification” in the present disclosure includes, but is not limited to, modification by methoxy, modification by fluoro and connection by thiophosphate group.

In the present disclosure, r indicates the following structural moiety:

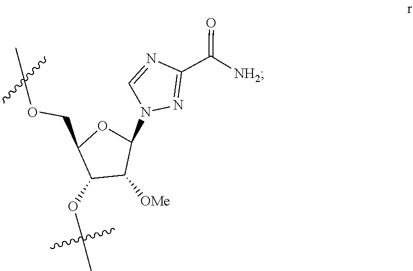 [SMILES: *OC[C@H]1O[C@@H](n2cnc(C(N)=O)n2)[C@H](OC)[C@@H]1O*]

r is the residue of r′, and r and other nucleotide residues are connected to each other by phosphate group or thiophosphate group, such as “ar” means that the residues a and r are connected by thiophosphate group, “ar” means that the residues a and r are connected by phosphate group.

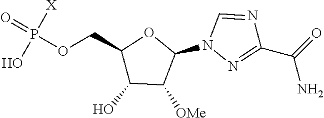

The r′ of the present disclosure is (wherein, X is selected from SH and OH), which is an analog of a natural nucleotide base, different from the natural nucleotide base of any published patent, and brings unexpected activity by the introduction in nucleotide sequence, and r′ is connected to other nucleotide residues in the nucleotide sequence in the form of r. The experimental results in the present disclosure show that the introduction of the nucleotide base analog makes the siRNA activity superior to that of the comparative compound “AD-66810”.

In the present disclosure, “embed” means that an embedded group is connected to at least one nucleotide residue in the sequence, including the replacement of a nucleotide residue with an embedded group (e.g., r) in the sequence.

In the present disclosure, the “embedded group” is a residue of an analog of a natural nucleotide base, which is different from the natural nucleotide base of any published patent, and after the embedded group is introduced into a nucleotide sequence, the sequence can have a certain function (such as unpredictable activity). In the present disclosure, the r is embedded into the oligonucleotide sequence as an embedded group, the r is able to inhibit the expression of genes, thereby producing unexpected activities.

In the present disclosure, “oligonucleotide embedded group” means that an embedded group is connected to at least one nucleotide residue in the oligonucleotide, including the replacement of a nucleotide residue with an embedded group (e.g., r) in the oligonucleotide.

In the present disclosure, r′-embedded sequence means that at least one nucleotide residue in the sequence is connected to r, including the replacement of a nucleotide residue with r in the sequence. In the present disclosure, the r′-embedded sequence can also be optionally modified, such as modification by methoxy, modification by fluoro and connection by phosphorothioate group. In the present disclosure, the r′-embedded sequences include, but are not limited to: r′-embedded siRNA, r′-embedded sense strands, and r′-embedded antisense strands. For example, 5′-aGUrrA·C-3′, 5′-rGgAAC-3′ and 5′-AG·UrAAcCuCr-3′ all belong to the case of r′-embedding.

In the present disclosure, the terms “complementary” or “reverse complementary” can be used interchangeably, and have a well-known meaning to those skilled in the art, that is, in a double-stranded nucleotide molecule, the base of one strand is matched with the base of the other strand in a complementary manner. Purine base adenine (A) always matches pyrimidine base uracil (U); purine base guanine (C) always matches the pyrimidine base cytosine (G). Each base pair comprises a purine and a pyrimidine. When adenine on one chain is always matched with uracil on the other chain, and guanine is always matched with cytosine, the two chains are considered to complement each other, and the sequence of this chain can be inferred from the sequence of its complementary chain. Accordingly, “mismatch” in this field means that in double-stranded nucleic acid, the bases at the corresponding positions are not matched in a complementary form.

In the present disclosure, unless otherwise specified, substantially reverse complementary refers to the existence of no more than 3 base mismatches between two nucleotide sequences; the essentially reverse complementary refers to the existence of no more than 1 base mismatch between the two nucleotide sequences; fully reverse complementary means that there are no mismatches between the two nucleotide sequences.

In the present disclosure, a “nucleotide difference” between one nucleotide sequence and another nucleotide sequence refers to a change in the base type of a nucleotide at the same position in the former compared to the latter, for example, when one nucleotide base in the latter is A, and the corresponding nucleotide base at the same position in the former is U, C, G, or R, it is assumed that there is a nucleotide difference between the two nucleotide sequences at that position. In some embodiments, when a base-free nucleotide or its equivalent is substituted for a nucleotide at the original position, a nucleotide difference may also be considered to have occurred at that position.

In the present disclosure, especially when describing the preparation method of the conjugated molecule or the preparation method of the siRNA conjugate of the present disclosure, unless otherwise specified, the nucleoside or nucleoside analogue monomer (nucleoside monomer) refers to, according to the type and sequence of nucleotides or nucleotide analogs in the siRNA or siRNA conjugate to be prepared, modified or unmodified nucleoside or phosphoramidite monomers of nucleoside analogue (unmodified or modified RNA phosporamidites, sometimes RNA phosphoramidites are also called Nucleoside phosphoramidites) used in solid phase synthesis of phosphoramidites. Solid phase synthesis of phosphoramidite is a well-known method for RNA synthesis to those skilled in the art. The nucleoside monomers used in the present disclosure are all commercially available. r′ and r are obtained by chemical synthesis.

The compounds of the present disclosure may exist in specific geometric or stereoisomeric forms. The present disclosure contemplates all such compounds, including (R)- and (S)-enantiomers, diastereoisomer and racemic mixtures thereof and other mixtures thereof, such as enantiomers or diastereoisomer enriched mixtures, all of which are within the scope of the present disclosure. Additional asymmetric carbon atoms may be present in substituents such as alkyl. All these isomers and their mixtures are included within the scope of the present disclosure.

Unless otherwise specified, the term “enantiomer” or “optical isomer” refers to stereoisomers that are mirror images of each other.

Unless otherwise specified, the term “diastereomer” refers to a stereoisomer in which a molecule has two or more chiral centers and the relationship between the molecules is not mirror images.

Unless otherwise specified, the absolute configuration of a stereogenic center is represented by a wedged solid bond ( [symbol] ) and a wedged dashed bond ( [symbol] ), and the relative configuration of a stereogenic center is represented by a straight solid bond ( [symbol] ) and a straight dashed bond ( [symbol] ), a wave line ( [symbol] ) is used to represent a wedged solid bond ( [symbol] ) or a wedged dashed bond ( [symbol] ), or the wave line ( [symbol] ) is used to represent a straight solid bond ( [symbol] ) or a straight dashed bond ( [symbol] ).

Unless otherwise specified, the terms “enriched in one isomer”, “enriched in isomers”, “enriched in one enantiomer” or “enriched in enantiomers” refer to the content of one of the isomers or enantiomers is less than 100%, and the content of the isomer or enantiomer is greater than or equal to 60%, or greater than or equal to 70%, or greater than or equal to 80%, or greater than or equal to 90%, or greater than or equal to 95%, or greater than or equal to 96%, or greater than or equal to 97%, or greater than or equal to 98%, or greater than or equal to 99%, or greater than or equal to 99.5%, or greater than or equal to 99.6%, or greater than or equal to 99.7%, or greater than or equal to 99.8%, or greater than or equal to 99.9%.

Unless otherwise specified, the term “isomer excess” or “enantiomer excess” refers to the difference between the relative percentages of two isomers or two enantiomers. For example, if the content of one isomer or enantiomer is 90%, and the content of the other isomer or enantiomer is 10%, the isomer or enantiomer excess (ee value) is 80%.

Optically active (R)- and (5)-isomer, or D and L isomer can be prepared using chiral synthesis or chiral reagents or other conventional techniques. If one kind of enantiomer of certain compound of the present disclosure is to be obtained, the pure desired enantiomer can be obtained by asymmetric synthesis or derivative action of chiral auxiliary followed by separating the resulting diastereomeric mixture and cleaving the auxiliary group. Alternatively, when the molecule contains a basic functional group (such as amino) or an acidic functional group (such as carboxyl), the compound reacts with an appropriate optically active acid or base to form a salt of the diastereomeric isomer which is then subjected to diastereomeric resolution through the conventional method in the art to obtain the pure enantiomer. In addition, the enantiomer and the diastereoisomer are generally isolated through chromatography which uses a chiral stationary phase and optionally combines with a chemical derivative method (such as carbamate generated from amine). The compound of the present disclosure may contain an unnatural proportion of atomic isotope at one or more than one atom(s) that constitute the compound. For example, the compound can be radiolabeled with a radioactive isotope, such as tritium (³H), iodine-125 (¹²⁵I) or C-14 (¹⁴C). For another example, deuterated drugs can be formed by replacing hydrogen with deuterium, the bond formed by deuterium and carbon is stronger than the bond formed by ordinary hydrogen and carbon, compared with non-deuterated drugs, deuterated drugs have the advantages of reduced toxic and side effects, increased drug stability, enhanced efficacy, extended biological half-life of drugs, etc. All isotopic variations of the compound of the present disclosure, whether radioactive or not, are encompassed within the scope of the present disclosure.

The term “salt” refers to a salt of the compound of the present disclosure that is prepared by reacting the compound having a specific substituent of the present disclosure with a relatively non-toxic acid or base. When the compound of the present disclosure contains a relatively acidic functional group, a base addition salt can be obtained by bringing the neutral form of the compound into contact with a sufficient amount of base in a pure solution or a suitable inert solvent. The pharmaceutically acceptable base addition salt includes a salt of sodium, potassium, calcium, ammonium, organic amine or magnesium, or similar salts. When the compound of the present disclosure contains a relatively basic functional group, an acid addition salt can be obtained by bringing the neutral form of the compound into contact with a sufficient amount of acid in a pure solution or a suitable inert solvent. Examples of the pharmaceutically acceptable acid addition salt include an inorganic acid salt, wherein the inorganic acid includes, for example, hydrochloric acid, hydrobromic acid, nitric acid, carbonic acid, bicarbonate, phosphoric acid, monohydrogen phosphate, dihydrogen phosphate, sulfuric acid, hydrogen sulfate, hydroiodic acid, phosphorous acid, and the like; and an organic acid salt, wherein the organic acid includes, for example, acetic acid, propionic acid, isobutyric acid, maleic acid, malonic acid, benzoic acid, succinic acid, suberic acid, fumaric acid, lactic acid, mandelic acid, phthalic acid, benzenesulfonic acid, p-toluenesulfonic acid, citric acid, tartaric acid, and methanesulfonic acid, and the like; and salts of amino acid (such as arginine and the like), and a salt of an organic acid such as glucuronic acid and the like. Certain specific compounds of the present disclosure contain both basic and acidic functional groups, thus can be converted to any base or acid addition salt.

The salt of the present disclosure can be prepared from the parent compound that contains an acidic or basic moiety by conventional chemical method. Generally, such salt can be prepared by reacting the free acid or base form of the compound with a stoichiometric amount of an appropriate base or acid in water or an organic solvent or a mixture thereof.

The compounds of the present disclosure can be prepared by a variety of synthetic methods known to those skilled in the art, including the specific embodiments listed below, the embodiments formed by their combination with other chemical synthesis methods, and equivalent alternatives known to those skilled in the art, preferred implementations include but are not limited to the embodiments of the present disclosure.

The structure of the compounds of the present disclosure can be confirmed by conventional methods known to those skilled in the art, and if the present disclosure relates to an absolute configuration of a compound, then the absolute configuration can be confirmed by means of conventional techniques in the art. For example, in the case of single crystal X-ray diffraction (SXRD), the absolute configuration can be confirmed by collecting diffraction intensity data from the cultured single crystal using a Bruker D8 venture diffractometer with CuKα radiation as the light source and scanning mode: φ/ω scan, and after collecting the relevant data, the crystal structure can be further analyzed by direct method (Shelxs97).

The solvent used in the present disclosure is commercially available.

Unless otherwise specified, the solvent ratios used in the column chromatography and preparative thin-layer silica gel chromatography of the present disclosure are all volume ratios.

### List of Abbreviations

| | |
|---|---|
| Ac | Acetyl |
| DMSO | Dimethyl sulfoxide |
| DMT/DMTr | 4,4′-Dimethoxytriphenylmethyl |
| dsRNA | Double-stranded ribonucleic acid |
| EC50 | Half maximum effect concentration |
| EDTA | Ethylenediaminetetraacetic acid disodium salt |
| i-Pr | isopropyl |
| Me | methyl |
| p-HPLC | Preparative high performance liquid chromatography for |
| | purification of compounds |
| RNA | Ribonucleic acid |
| RNAi | Ribonucleic acid interference technology |
| siRNA | Small interfering ribonucleic acid |
| Tris | Trihydroxymethyl aminomethane |

The compounds of the present disclosure are named according to the conventional naming principles in the art or by ChemDraw® software, and the commercially available compounds use the supplier catalog names.

The positive progressive effect of the present disclosure is that the siRNA conjugate of the present disclosure can be used to prepare double-stranded siRNA conjugate, and the double-stranded siRNA conjugate can effectively reduce the S antigen content and E antigen content of the hepatitis B virus, providing an effective, feasible approach for the functional cure of chronic hepatitis B.

### DETAILED DESCRIPTION OF THE PREFERRED EMBODIMENT

The present disclosure is described in detail by the embodiments below, but it does not mean that there are any adverse restrictions on the present disclosure. The compounds of the present disclosure can be prepared by a variety of synthetic methods known to those skilled in the art, including the specific embodiments listed below, the embodiments formed by their combination with other chemical synthesis methods, and equivalent alternatives known to those skilled in the art, preferred embodiments include but are not limited to the embodiments of the present disclosure. It will be apparent to those skilled in the art that various variations and improvements can be made to specific embodiments of the present disclosure without departing from the spirit and scope of the present disclosure.

The intermediate compound L96 was prepared with reference to Nair, J. K. et al. J. Am. Chem. Soc. (2014), 136, 16958-16961.

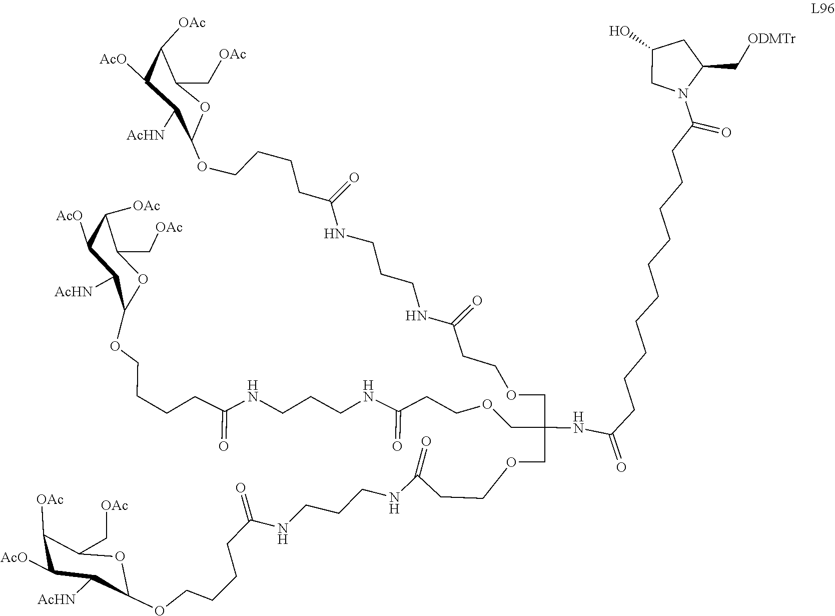

### Embodiment 1 Synthesis of Phosphoramidite Monomer for Introducing Nucleotide Analogue r into Biological Oligomer

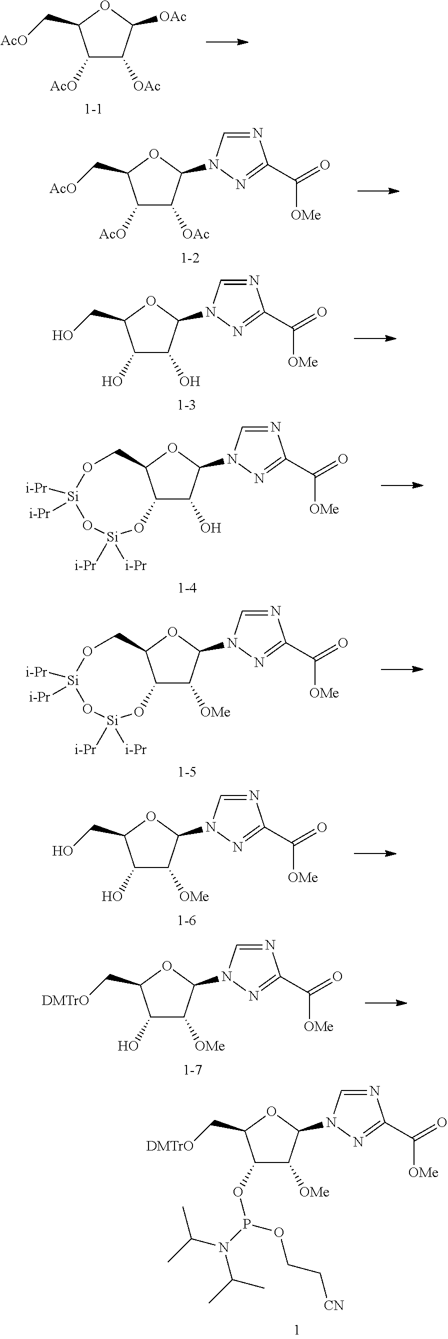

Step A: (2S,3R,4R,5R, 6R)-3-Acetamido-6-(acetoxymethyl)tetrahydro-2H-pyran-2,4,5-triyl triacetate (i.e., formula 1-1) (30 g, 94.26 mmol) and methyl 1,2,4-triazole-3-carboxylate (11.98 g, 94.26 mmol) were dissolved in a mixture solution of methyl acetate (220 mL), and the mixture was concentrated in a 90° C. oil bath at 1 bar until the mixture was nearly completely dried. A methyl acetate solution (2 mL) of trifluoromethanesulfonic acid (141.46 mg, 0.94 mmol) was added to the mixture and stirred at 30 mbar in an oil bath at 125° C. for 4 hours. The reaction solution was cooled to 70° C., and ethanol (70 mL) was added, then the mixture was stirred at 70° C. until a uniform solution was formed, and the stirring was stopped and the mixture was cooled to 50° C. After the precipitate was formed, the mixture was cooled to 25° C. and left at 0° C. for 16 hours. The reaction solution was filtered through a Buchner funnel, and the filter cake was rinsed with 180 mL (60 mL×3) of ethanol, and dried in vacuum to obtain 1-2. ¹H NMR (400 MHz, CDCl₃): δ 8.40 (s, 1H), 6.04 (d, J=3.42 Hz, 1H), 5.69-5.81 (m, 1H), 5.54 (t, J=5.38 Hz, 1H), 4.42-4.51 (m, 2H), 4.16-4.30 (m, 1H), 3.98 (s, 3H), 2.05-2.18 (m, 9H).

Step B: The compound as shown in formula 1-2 (15 g, 38.93 mmol) and triethylamine (4.14 g, 40.87 mmol) were dissolved in methanol (100 mL). The mixture was stirred at 50° C. for 17 hours under the protection of nitrogen. The reaction solution was concentrated under reduced pressure to obtain 1-3. ¹H NMR (400 MHz, CD₃OD): δ 8.87 (s, 1H), 5.93 (d, J=3.42 Hz, 1H), 4.48 (dd, J=3.48, 4.83 Hz, 1H), 4.33 (t, J=5.26 Hz, 1H), 4.10-4.16 (m, 1H), 3.95 (s, 3H), 3.84 (dd, J=3.24, 12.29 Hz, 1H), 3.70 (dd, J=4.46, 12.29 Hz, 1H).

Step C: The compound as shown in formula 1-3 (10 g, 38.58 mmol) was dissolved in pyridine (250 mL), and 1,3-dichloro-1,1,3,3-tetraisopropyldisiloxane (12.29 g, 38.97 mmol) was added dropwise at 0° C. The mixture was gradually warmed to 25° C. and stirred for 16 hours. The reaction solution was concentrated under reduced pressure, suspended in ethyl acetate (250 mL), and filtered through a Buchner funnel. The filtrate was washed with 750 mL of 3 M hydrochloric acid (250 mL×3) and 250 mL of saturated brine (250 mL×1), dried over anhydrous sodium sulfate, filtered and concentrated under reduced pressure to obtain the crude product. The crude product was purified by column chromatography (SiO₂, petroleum ether/dichloromethane/ethyl acetate=3/1/1) to obtain 1-4. ¹H NMR (400 MHz, CDCl₃): δ 8.43 (s, 1H), 5.95 (s, 1H), 4.73 (dd, J=4.75, 8.00 Hz, 1H), 4.41 (d, J=4.75 Hz, 1H), 4.09-4.19 (m, 2H), 3.94-4.03 (m, 4H), 2.71-3.34 (m, 1H), 1.01-1.15 (m, 28H).

Step D: Methyl iodide (11.64 g, 82.02 mmol) was added to a mixed N,N-dimethylformamide (50 mL) solution of the compound as shown in formula 1-4 (8.23 g, 16.40 mmol), potassium carbonate (11.34 g, 82.02 mmol) and silver (I) oxide (19.01 g, 82.02 mmol), and the mixture was stirred at 25° C. for 3 hours. The reaction solution was diluted with ethyl acetate (300 mL) and filtered through a Buchner funnel. The filtrate was washed with 250 mL (250 mL×1) of sodium thiosulfate aqueous solution, 250 mL (250 mL×1) of water and 250 mL (250 mL×1) of brine, dried with anhydrous sodium sulfate, filtered and concentrated under reduced pressure to obtain the crude product. The mixture was purified by column chromatography (SiO₂, petroleum ether/ethyl acetate=5/1) to obtain 1-5. ¹H NMR (400 MHz, CDCl₃): δ 8.58 (s, 1H), 5.91 (s, 1H), 4.46 (dd, J=4.22, 9.35 Hz, 1H), 4.17-4.28 (m, 2H), 3.96-4.06 (m, 5H), 3.68 (s, 3H), 0.99-1.13 (m, 28H).

Step E: Triethylamine trihydrofluoride (2.25 g, 13.95 mmol) was added to a tetrahydrofuran (50 mL) solution of the compound as shown in formula 1-5 (3.27 g, 6.34 mmol) dropwise at 0° C., and the mixture was gradually warmed to 25° C. and stirred for 16 hours. The reaction solution was concentrated under reduced pressure to obtain the crude product. The crude product was purified by column chromatography (SiO₂, dichloromethane/methanol=20/1) to obtain 1-6. ¹H NMR (400 MHz, CD₃OD): δ 8.88 (s, 1H), 6.04 (d, J=3.26 Hz, 1H), 4.44 (t, J=5.33 Hz, 1H), 4.20 (dd, J=3.33, 4.83 Hz, 1H), 4.07-4.14 (m, 1H), 3.96 (s, 3H), 3.84 (dd, J=3.20, 12.36 Hz, 1H), 3.69 (dd, J=4.39, 12.30 Hz, 1H), 3.52 (s, 3H).

Step F: 4,4-Dimethoxytrityl chloride (2.42 g, 7.14 mmol) was added to a pyridine (20 mL) solution of the compound as shown in formula 1-6 (1.30 g, 4.76 mmol) at 0° C., and the mixture was stirred at 25° C. for 16 hours. The reaction solution was diluted with ethyl acetate (70 mL), quenched with saturated sodium bicarbonate aqueous solution (20 mL) at 25° C. and diluted with water (40 mL). After the phases were separated, the combined organic phases were washed with 60 mL of water (60 mL×1) and 60 mL of brine (60 mL×1), dried with anhydrous sodium sulfate, filtered and concentrated under reduced pressure to obtain the crude product. The crude product was purified by p-HPLC (separation column: Phenomenex luna C18 (size: 250 mm×50 mm, particle size: 10 μm); mobile phase: [water (10 mM ammonium bicarbonate)-acetonitrile]; elution gradient: 35%-65%, 20 min) to obtain 1-7. ¹H NMR (400 MHz, CDCl₃): δ 8.44 (s, 1H), 7.38-7.45 (m, 2H), 7.28-7.34 (m, 5H), 7.18-7.27 (m, 2H), 6.70-6.92 (m, 4H), 5.97 (d, J=2.88 Hz, 1H), 4.37-4.43 (m, 1H), 4.33 (dd, J=2.88, 5.00 Hz, 1H), 4.19-4.25 (m, 1H), 3.98 (s, 3H), 3.80 (s, 6H), 3.58 (s, 3H), 3.43-3.49 (m, 1H), 3.33-3.40 (m, 1H), 2.55 (d, J=6.88 Hz, 1H). LCMS (ESI) m/z: 574.2 [M−H]⁻.

Step G: 2-Cyanoethyl-N,N-diisopropylchlorophosphonamide (678.45 mg, 2.87 mmol) and N,N-diisopropylethylamine were added to a dichloromethane (8 mL) solution of the compound as shown in formula 1-7 (1.10 g, 1.91 mmol) at 0° C., and the mixture was stirred for 0.5 hours at 20° C. The reaction solution was concentrated under reduced pressure to obtain the crude product. The crude product was purified by column chromatography (SiO₂, petroleum ether/ethyl acetate=50/1 to 1/2) to obtain the compound as shown in formula 1. LCMS (ESI) m/z: 776.3 [M+H]⁺.

RNA synthesis: Oligonucleotides were synthesized according to the solid phase synthesis technology of phosphoramidite. Synthesis was carried out on a solid support made of controllable porous glass (CPG, 500 Å). All 2′-modified RNA phosphoramidite and auxiliary reagents are commercially available. All amides were dissolved in anhydrous acetonitrile and molecular sieves (3 Å) were added, and the coupling time was 5 min using 5-ethylthio-1H-tetrazole (ETT) as the activator. Thiophosphate bond was generated by using a 50 mM anhydrous acetonitrile/pyridine (v/v=1/1) solution of 3-((dimethylamino-methylene)amino)-3H-1,2,4-dithiazole-3-thione (DDTT), the reaction solution was reacted for 3 minutes. All sequences were synthesized after the DMT group was finally removed.

Cleavage and deprotection of CPG-bound oligomers: After the solid phase synthesis was terminated, the protecting groups were removed by treating with an acetonitrile solution of 20% diethylamine for 30 minutes, without cleavage of oligonucleotides from CPG. Subsequently, the dried CPG was treated with concentrated ammonia water at 40° C. for 18 hours. After centrifugation, the supernatant was transferred to a new tube and CPG was washed with ammonia water. The combined solutions were concentrated to obtain a solid mixture.

Purification of oligonucleotides: Oligomer was purified by anion exchange HPLC using NanoQ. Buffer A was 10 mM sodium perchlorate solution, 20 mM Tris, 1 mM EDTA, pH 7.4 and containing 20% acetonitrile, and Buffer B was 500 mM sodium perchlorate, 20 mM Tris, 1 mM EDTA, pH 7.4 and containing 20% acetonitrile. The target product was separated and desalted by reversed-phase C18 column.

Annealing of oligoribonucleotides to generate siRNA: The RNA oligomers to be annealed were prepared to 200 μM in sterile RNase Free H₂O (without RNA hydrolase). The annealing reaction system was set up as follows, a total volume of 100 μL of the mixture, 10 nmol, was placed in a 95° C. water bath for 10 minutes (100 nmol required high temperature for 20 minutes)—quickly put into a 60° C. water bath to cool down naturally—after annealing was completed, the solution should not be stored at high temperature. Complementary strands were mixed by combining equimolar RNA solutions.

TABLE 1
Core sequences of dsRNA targeting hepatitis B virus genes and their
modified counterparts

| Core sequence | | | | Modified sequence | | | |
|---|---|---|---|---|---|---|---|
| | Sense | | Antisense | | Sense | | Antisense |
| | strand | | strand | | strand | | strand |
| SEQ ID | sequence | SEQ ID | sequence | SEQ ID | sequence | SEQ ID | sequence |
| NO | (5′-3′) | NO | (5′-3′) | NO | (5′-3′) | NO | (5′-3′) |
| 1 | GUGUGCA | 3 | UGUGAA | 5 | g•u•guGcA | 7 | u•G•ugaAg |
| | CUUCGCU | | GCGAAGU | | CUucgcuuc | | CGaaguGc |
| | UCACA | | GCACAC | | acaL* | | Acac•u•u |
| 1 | GUGUGCA | 3 | UGUGAA | 9 | g•r•guGcA | 7 | u•G•ugaAg |
| | CUUCGCU | | GCGAAGU | | CUucgcuuc | | CGaaguGc |
| | UCACA | | GCACAC | | acaL | | Acac•u•u |
| 1 | GUGUGCA | 3 | UGUGAA | 10 | g•u•ruGcA | 7 | u•G•ugaAg |
| | CUUCGCU | | GCGAAGU | | CUucgcuuc | | CGaaguGc |
| | UCACA | | GCACAC | | acaL | | Acac•u•u |
| 1 | GUGUGCA | 3 | UGUGAA | 9 | g•r•guGcA | 11 | u•G•ugargC |
| | CUUCGCU | | GCGAAGU | | CUucgcuuc | | GaaguGcAc |
| | UCACA | | GCACAC | | acaL | | ac•u•u |
| 2 | CACCAUG | 4 | AGGUGA | 6 | c•r•ccauGc | 8 | a•G•gugAa |
| | CAACUUU | | AAAAGU | | AACuuuuu | | AAaguuGc |
| | UUCACCU | | UGCAUGG | | caccuL | | Auggug•u•u |
| | | | UG | | | | |
| 2 | CACCAUG | 4 | AGGUGA | 12 | c•a•ccauGc | 8 | a•G•gugAa |
| | CAACUUU | | AAAAGU | | AACuuuuu | | AAaguuGc |
| | UUCACCU | | UGCAUGG | | caccuL | | Auggug•u•u |
| | | | UG | | | | |

*: L is the residue of small molecule segment L96 after chemical reaction, which is bound to nucleic acid by covalent bond, and the structure thereof is shown in the following formula.

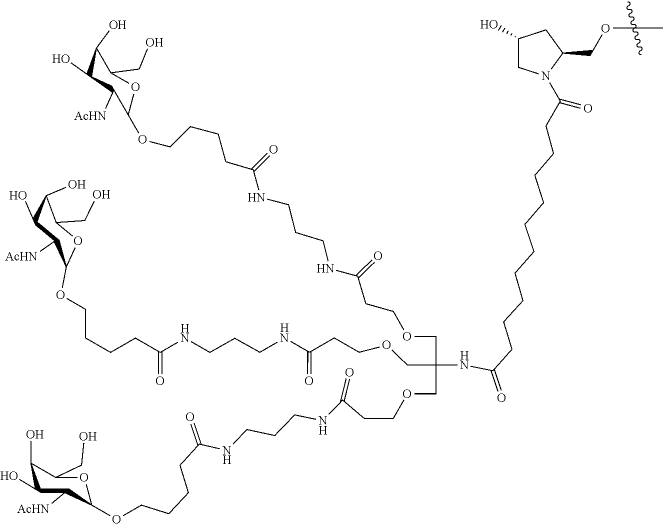

### Experimental Embodiment 2 HBV Test In Vitro

Experimental Purpose:

The content of HBV antigens (HBsAg and HBeAg) in the culture supernatant of HepG2-NTCP cells was detected by enzyme linked immunosorbent assay (ELISA), and the EC₅₀ value of the compound was used as an indicator to evaluate the inhibitory activity of siRNA conjugates on HBV; at the same time, Cell-titer Glo assay was used to evaluate the cell viability to evaluate the cytotoxicity of siRNA conjugates.

Experimental Materials:

Cell line: HepG2-NTCP cells.

HepG2-NTCP cell culture medium (DMEM, Invitrogen-11330032; 10% serum, Invitrogen-10099141; 100 units/mL penicillin and 100 μg/mL streptomycin, Hyclone-SV30010; 1% non-essential amino acids, Invitrogen-11140050; 2 mM L-Glutamine, Invitrogen-25030081; 1 mM sodium pyruvate, Gibco-11360-070; 500 μg/mL Geneticin, Invitrogen-10131027).

Reagents: Trypsin (Invitrogen-25300062); DPBS (Corning-21031CVR); DMSO (Sigma-D2650-100 mL); Cell-titer Glo (Promega-G7573); hepatitis B surface antigen quantitative detection kit (Antu Bio-CL 0310); hepatitis B e antigen quantitative detection kit (Antu Bio-CL 0312).

Consumables and instruments: 96-well cell culture plate (Corning-3599); CO₂ incubator (HERA-Cell-240); enzyme labeling instrument (BioTek Synergy 2).

Experimental Steps and Methods:

On day 0, HepG2-NTCP (7.5×10⁴ cells/well) cells were seeded into 48-well plates and cultured overnight at 37° C., 5% CO₂.

On day 1, the culture medium containing 1% DMSO was replaced.

On day 2, HepG2-NTCP (2000 GE/cell) was infected with D-type HBV (concentrated from HepG2.2.15 cell culture supernatant).

On day 3, the infection solution was aspirated and fresh culture medium containing 1% DMSO was added.

On day 6, siRNA conjugates were transfected according to the instructions of Lipofectamine® RNAiMax (Invitrogen). The conjugate was diluted by 5-fold gradient with 7 concentrations in triplicate wells, and the final concentration was 6.4 pM. The conjugate was a combination of sense and antisense strands as a single chemical entity with a maximum concentration of 100 nM.

On day 12, the supernatants in the culture wells were collected, and HBV surface antigen and e antigen were determined by ELISA. After the supernatant was collected, Cell-titer Glo was added to measure cell viability.

ELISA was used to determine hepatitis B virus surface antigen (HBsAg) and e antigen (HBeAg), the specific steps referred to the product manual and the steps were briefly described as follows: 50 μL of the sample and standard substance were added to the reaction plate respectively, and then 50 μL of enzyme conjugate was added to each well, and the mixture was shaked and mixed, incubated at 37° C. for 60 minutes, then the plate was washed for 5 times, then 50 μL of luminescent substrate was added to each well, and the mixture was mixed well, reacted at room temperature for 10 minutes in the dark, and finally the chemiluminescence intensity was detected by microplate reader.

Data Analysis:

a. The percentage of cell viability was calculated:

Viability %=(luminescence value of sample−luminescence value of culture medium)/(luminescence value of DMSO control−luminescence value of culture medium control)×100.

b. The percentage inhibition of HBV surface antigen and e antigen were calculated:

Inh %.=(1−antigen value in sample/DMSO control antigen value)×100.

c. CC₅₀ and EC₅₀ were calculated:

CC₅₀ and 50% inhibitory concentration (EC₅₀) values for compounds against HBV were calculated using GraphPad Prism software.

4. Experimental results: see Table 2.

TABLE 2
Experimental results in reducing HBsAg and HBeAg
levels in cells by the test sequences

| Test sequence | | | | Experimental | | |
|---|---|---|---|---|---|---|
| | Sense | | Antisense | results | | |
| | strand | | strand | HBsAg | HBeAg | Cell |
| SEQ ID | sequence | SEQ ID | sequence | EC₅₀ | EC₅₀ | Viability |
| NO | (5′-3′) | NO | (5′-3′) | (pM) | (pM) | CC₅₀ (nM) |
| 5 | g•u•guGcACUu | 7 | u•G•ugaAgCGaa | 25.26 | 238.1 | >2.5 |
| | cgcuucacaL* | | guGcAcac•u•u | | | |
| 9 | g•r•guGcACUu | 7 | u•G•ugaAgCGaa | 24.12 | 38.28 | >2.5 |
| | cgcuucacaL | | guGcAcac•u•u | | | |
| 10 | g•u•ruGcACUu | 7 | u•G•ugaAgCGaa | 39.49 | 67.82 | >2.5 |
| | cgcuucacaL | | guGcAcac•u•u | | | |
| 9 | g•r•guGcACUu | 11 | u•G•ugargCGaa | 11.92 | 139.50 | >2.5 |
| | cgcuucacaL | | guGcAcac•u•u | | | |
| 12 | c•a•ccauGcAA | 8 | a•G•gugAaAAag | 148.40 | 51.86 | >6.25 |
| | CuuuuucaccuL | | uuGcAuggug•u• | | | |
| | | | u | | | |
| 6 | c•r•ccauGcAA | 8 | a•G•gugAaAAag | 67.35 | 33.22 | >6.25 |
| | CuuuuucaccuL | | uuGcAuggug•u• | | | |
| | | | u | | | |

In Table 2, the dsRNA consisting of SEQ ID NO: 5 and SEQ ID NO: 7, the dsRNA consisting of SEQ ID NO: 12 and SEQ ID NO: 8 are recorded in WO2018/195165A1.

Conclusion: The embodiment of the present disclosure shows unexpected excellent inhibitory activity against HBsAg and HBeAg, which demonstrates that the vitality of hepatitis B virus can be inhibited. Using r as an oligonucleotide embedded group is expected to improve the silencing activity and persistence of oligonucleotides in animal models, and decrease the risk of off-target by weakening the binding to potential off-target genes. At present, the commonly clinical safety risk of oligonucleotides is liver toxicity caused by off-target, and using r as the embedded group of oligonucleotide is expected to provide an efficient and safe treatment means for clinically functional cure of chronic hepatitis B.

1. A use of r as an oligonucleotide embedded group, and the r 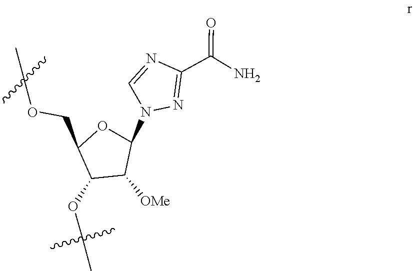
wherein, the oligonucleotide is a nucleotide sequence containing 10 to 50 nucleotides or nucleotide base pairs, and the oligonucleotide is able to inhibit or block gene expression.

2. The use as claimed in claim 1, wherein the gene is HBV gene.

3. The use as claimed in claim 1, wherein, the oligonucleotide is siRNA.

4. The use as claimed in claim 3, wherein, the siRNA comprises sense strand and antisense strand.

5. The use as claimed in claim 4, wherein, the r is only embedded in the sense strand of the siRNA.

6. The use as claimed in claim 4, wherein, the r is only embedded in the antisense strand of the siRNA.

7. The use as claimed in claim 4, wherein, the r is embedded in the sense strand and antisense strand of the siRNA.

8. The use as claimed in claim 4, wherein, the sense strand of the siRNA comprises a sequence as shown in SEQ ID NO: 5 or SEQ ID NO: 12.

9. The use as claimed in claim 4, wherein,
the antisense strand of the siRNA comprises a sequence as shown in SEQ ID NO: 7 or SEQ ID NO: 8.

10. The use as claimed in claim 4, wherein,
the sense strand and the antisense strand of the siRNA respectively comprises a sequence as shown in SEQ ID NO: 5 and SEQ ID NO: 7, or the sense strand and the antisense strand of the siRNA respectively comprises a sequence as shown in SEQ ID NO: 12 and SEQ ID NO: 8.

11. A siRNA conjugate, wherein, the structure thereof is shown in formula (I):
S-L (I)
wherein, the nucleotide sequence of S is shown in SEQ ID NO: 6, SEQ ID NO: 9 or SEQ ID NO: 10, and the L is shown in formula (II): 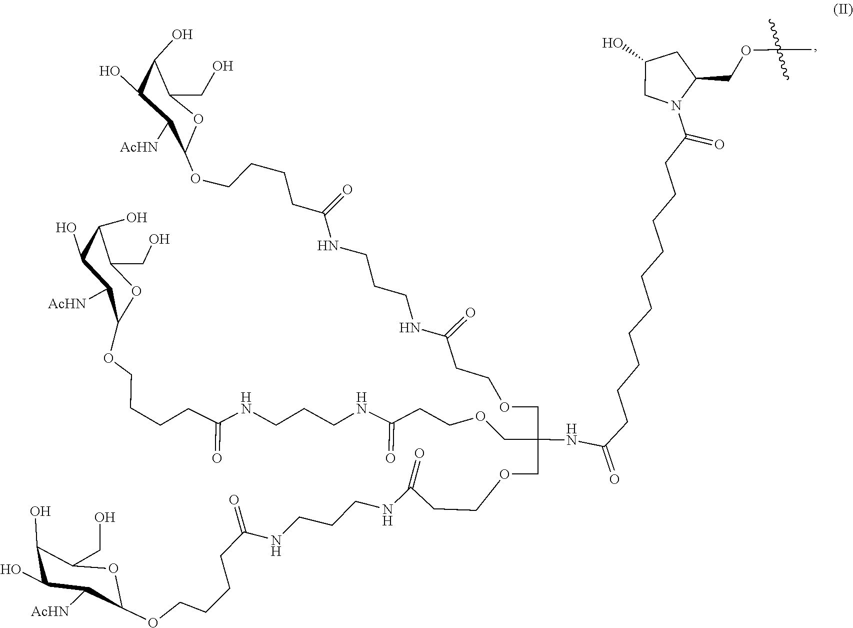
and the L is connected to the 3′ end of the nucleotide sequence of the S.

12. The siRNA conjugate as claimed in claim 11, wherein the thiophosphate moiety of the siRNA conjugate comprises (R)- and (S)-enantiomers, diastereomers, or racemic mixtures thereof.

13. A salt of the siRNA conjugate as defined in claim 11.

14. A double-stranded siRNA conjugate, wherein, the double-stranded siRNA conjugate comprises a sense strand and an antisense strand, and the sense strand is the siRNA conjugate as defined in claim 11.

15. The double-stranded siRNA conjugate as claimed in claim 14, wherein, the nucleotide sequence of the antisense strand is shown in SEQ ID NO: 7, SEQ ID NO: 8 or SEQ ID NO: 11.

16. The double-stranded siRNA conjugate as claimed in claim 14, wherein, the thiophosphate moiety of the double-stranded siRNA conjugate comprises (R)- and (S)-enantiomers, diastereomers, or racemic mixtures thereof.

17. A salt of the double-stranded siRNA conjugate as defined in claim 14.

18. The salt as claimed in claim 13, wherein, the salt comprises a base addition salt and an acid addition salt.

19. The salt as claimed in claim 18, wherein, the base addition salt comprises a sodium, potassium, calcium, ammonium, organic amine or magnesium salt; or the acid addition salt comprises an inorganic acid salt and an organic acid salt.

20. The salt as claimed in claim 19, wherein, the inorganic acid comprises hydrochloric acid, hydrobromic acid, nitric acid, carbonic acid, bicarbonate, phosphoric acid, monohydrogen phosphate, dihydrogen phosphate, sulfuric acid, hydrogen sulfate, hydroiodic acid, phosphorous acid, and the organic acid comprises acetic acid, propionic acid, isobutyric acid, maleic acid, malonic acid, benzoic acid, succinic acid, suberic acid, fumaric acid, lactic acid, mandelic acid, phthalic acid, benzenesulfonic acid, p-toluenesulfonic acid, citric acid, tartaric acid, or methanesulfonic acid.

21. (canceled)
